(19) United States
(12) Patent Application Publication
(10) Pub. No.: US 2017/0151770 A1
(43) Pub. Date: Jun. 1, 2017

(54) PROCEDURES FOR THE PRODUCTION OF CLOTHING OR BANDAGES

(71) Applicant: NTT New Textile Technologies GmbH, Balingen (DE)

(72) Inventor: Hans Bauer, Balingen (DE)

(21) Appl. No.: 14/974,470

(22) Filed: Dec. 18, 2015

(30) Foreign Application Priority Data
Nov. 27, 2015 (EP) 15020237

(51) Int. Cl. B32B 37/14; A61F 13/00; A41C 3/00; B32B 38/00; A41C 5/00

(52) U.S. Cl. CPC B32B 37/14; B32B 38/0004; A41C 5/00; A41C 3/0014; A61F 13/00987; B32B 2437/00; B32B 2535/00; B32B 2305/72

(57) ABSTRACT

In a method for the manufacture of garments or bandages, a fabric layer is bonded with a composite of elastomer and reinforcement material, which is produced in that on an elastomer layer is applied the reinforcement material in liquid or soft, deformable state, which hardens after application.

### FIELD OF THE INVENTION

The present invention relates to the method of manufacture of garments or bandages.

### BACKGROUND OF THE INVENTION

DE 101 33 644 C2 discloses a method for manufacturing undergarments or sports garment, in which in a first process step an elastomer adhesive sheet is applied onto a fabric layer, which corresponds to the subsequent outer contour of the garment. In a second process step, the fabric is cut in the area of the elastomer adhesive sheet.

Various sections of the garment may be subject to different levels of stress, which should be considered when choosing the fabric layer. At the same time, however, a high level of comfort must be ensured, in particular in undergarments.

For further prior art, reference is made to the documents US 2002/0 106 970 A1 and EP 0 187 270 A1.

The invention has for its object to manufacture by simple measures a garment or bandage, which with high wearing comfort also withstands higher loads.

This object is achieved with the features of claim 1. The dependent claims specify expedient further developments.

The inventive process relates to the manufacture of garments such as undergarments or sportswear garments, such as bra, but also outer garments. Furthermore, also bandages such as joint bandages, knee or elbow bandages can be manufactured by the method according to the invention.

In the process, a fabric layer is connected to a combination or a composite of elastomer and reinforcement material. This combination is produced by applying a liquid or soft and deformable reinforcement material to an elastomer layer, which cures after the application.

The reinforcing material has a higher strength than the elastomer so that a composite of the elastomer and the reinforcing material is thus obtained, which has a significantly higher tensile strength than the elastomer without reinforcement material. The introduction of reinforcing elements such as metal bars or springs can be dispensed with.

The elastomer layer and the reinforcement material are applied by a respective nozzle. The position of each nozzle can be adjusted in all three spatial directions so that every position on the garment or bandage can be approached. The application of the elastomer and the reinforcement material on the garment or the bandage can be done by changing the position of the nozzle along a predetermined path and be carried out for example in strip form. The position adjustment of each nozzle is carried by control signals of a control device, wherein the control signals can be generated on the basis of a digital template.

Also, the quantity of the elastomer material and reinforcement material exiting the respective nozzle can be variably adjusted, in particular also by control signals of the control device on the basis of the digital template.

The nozzles for the application of the elastomer material and the reinforcement material can be equipped with its own separate adjustment mechanism. However, it is also possible to provide a common adjustment mechanism for both nozzles to control the nozzle for the application of the elastomer and then the nozzle for the application of the reinforcement material with the same adjustment mechanism. Optionally, thereafter another elastomer layer can be applied by the corresponding nozzle.

The reinforcement material may be applied in one layer or optionally in several layers, making it possible to arbitrarily form a reinforcing bead made of reinforcement material in terms of height or width (relative to its longitudinal axis) and/or the form. The at least two superposed layers of reinforcement material may differ in width and/or height, whereby layers of the same width and/or height of the reinforcement material are also possible.

The reinforcing bead formed from the cured reinforcement material has, with respect to its longitudinal axis, a symmetrical or non-symmetrical cross-section, which is reduced, for example in the direction of the rim region of the garment or bandage.

The reinforcement material is for example a plastic material which is applied to the elastomer layer in liquid or soft, deformable state. After curing, the reinforcement material has a higher tensile strength than the elastomer so that accordingly the composite of elastomer and reinforcement material has also the desired higher tensile strength.

Since the reinforcement material is applied in liquid or soft, deformable state, there are no restrictions with regard to the contour of the composite material or the combination of the elastomer and the reinforcement material. The elastomer is applied, for example, in tracks; the reinforcement material can be applied in such a way on the track-like elastomer layer that a lateral distance from the lateral rim regions of the elastomer exists. The track-like elastomer layer can extend straight or curved, where appropriate also kinked, and this course can be followed easily also by the reinforcement material to be applied.

Another advantage is that the quantity of the reinforcement material to be applied on the elastomer layer can be customized as needed. So it is possible, for example, to vary the quantity of the reinforcement material at different locations along the elastomer layer. Furthermore, it is also possible to apply a track of reinforcement material with a uniform thickness and uniform diameter. The track of reinforcement material on the elastomer layer is formed advantageously continuously.

The application of the reinforcement material can be carried out in a continuous straight or curved, possibly kinked track or, in a further advantageous embodiment, in an irregular but continuous application in which the reinforcement material extends for example in a meandering line with lateral and/or vertical bulges to the direction of application. The serpentine line achieves an additional spring effect in the reinforcement material.

The reinforcement material is advantageously completely surrounded by the elastomer. As a result, the wearing comfort is improved because the reinforcement material cannot come into direct contact with the wearer's skin.

The wrapping of the reinforcement material with elastomer can be carried out in that the reinforcement material is immersed during the application in the soft elastomer of the elastomer layer and hardens within the elastomer layer. According to an advantageous embodiment, it can also be expedient to apply a second layer of elastomer in a subsequent step onto the first elastomer layer, to which the reinforcement material is applied so that the reinforcement material is covered by the second elastomer layer. The first and second elastomer layers are preferably made of the same elastomer, which connects to a layer enveloping the reinforcement material. In this way, the reinforcement material is enveloped all around in elastomer.

According to yet another advantageous embodiment, the elastomer layer, which is the carrier of the reinforcement material, is applied directly to the fabric layer. The fabric layer itself has, in a preferred embodiment, an intrinsic elasticity or extensibility, which is advantageously higher than the extensibility of the cured reinforcement material. This ensures that the pulling forces are taken up in at least a significant proportion by the reinforcement material in the elastomer. At the same time, the elastomer connects with the fabric layer so that a firm connection between the fabric layer and elastomer develops. The elastomer embedded in the reinforcement material can be additionally connected to the fabric layer.

Alternatively to the application to a fabric layer, the combination or the composite of elastomer and reinforcement material can also be manufactured in that an elastomer layer is introduced into a molding and the reinforcement material is applied to the elastomer layer in the molding. After curing, one obtains in this way a cohesive arrangement which is formed by the combination of formed by elastomer and reinforcement material and can then be connected to the fabric layer. The connection is performed for example by gluing, in that for example the elastomer of the composite is bonded to the fabric layer, or in a mechanical way, in that the elastomer or the reinforcement material is bonded to the fabric layer, for example, by sewing or clipping or the like.

Both in the application directly to the fabric layer and in the manufacture in a molding, it is expedient that after the application of the reinforcement material, a second elastomer layer is applied the first elastomer layer so that the reinforcement material is completely embedded within the elastomer.

According to a further advantageous embodiment, the elastomer reinforced with the reinforcement material is provided with a flocking material, for example, natural, semisynthetic or synthetic fibers or flakes. The flocking can be applied in the not yet fully cured state of the elastomer so that after drying, the flocking material is bonded with the elastomer. The flocking increases the wearing comfort and, in addition, is also able to absorb moisture such as sweat.

As the elastomer, for example a thermoplastic elastomer is used, which is softened or liquefied upon heating and hardens with cooling. Accordingly, the elastomer is applied in the heated state as a layer, wherein the reinforcement material is advantageously applied on the still soft elastomer also in liquid or soft, deformable state.

The reinforcement material preferably has a higher melting point than the elastomer so that upon the application of the reinforcement material melts, partially or fully, the cured elastomer and the reinforcement material penetrates partially into the elastomer layer.

There are also embodiments possible in which the reinforcement material is applied onto the still soft elastomer, as well as embodiments in which the elastomer layer first hardens and only then the reinforcement material is applied.

If a second elastomer layer is applied, this is done either only after the hardening of the reinforcement material or, in an alternative embodiment, in the not yet fully cured state of the reinforcement material.

The suitable reinforcement material is especially a plastic material, such as PLA (polylactide) or ABS (acrylonitrile-butadiene-styrene copolymer). The melting point of the plastic material is for example 180° C. or 220° C.

The application of the plastic material is carried out for example using a 3-D printer head, which allows to apply the plastic material in any desired three-dimensional manner, for example, over the length, the width and/or height of the application. This makes it possible, for example, to apply a wider and/or higher layer onto more stressed areas and to apply a narrower and/or lower layer onto less stressed areas. Using the 3-D print head, the reinforcement material is applied in layers until the desired final shape of the reinforcement material on the elastomer layer has been reached.

The application of the plastic material can also be carried out with a spray device, for example, with a spray gun.

According to a further advantageous embodiment, a plurality of track-like elastomer layers is applied to the fabric layer, which is provided without reinforcement material. After application of the track-like elastomer layers, the fabric sheet is then cut out within the elastomer layers corresponding to the rim contour of the garment or the bandage to be manufactured. This makes it possible to manufacture an item of clothing or a bandage whose marginal contour is reinforced with the elastomer layer, whereas the inner portions of the fabric layer, spaced from the peripheral contour, is provided without continuous surface coating with elastomer and accordingly ensure an improved wearing comfort. The rim-side elastomer layer has a high elasticity and carrying capacity.

The track-like elastomer layers can either be applied to only a single-ply fabric layer or are used to connect two layers of fabric in such a manner that first an elastomer layer is applied to a fabric layer and then a second fabric layer is placed onto the first fabric layer.

Placing various track-like elastomer layers without reinforcement material onto the fabric layer can optionally be combined with an elastomer layer having incorporated reinforcement material therein. For example, it may be expedient for a garment to form a part of the rim contour of a track-like elastomer layer without reinforcement material and a part of an elastomer layer with reinforcement material. For example, in case of a brassiere, the support can be made from a composite of a track-like elastomer layers with incorporated reinforcement material. In the area of these elastomer layers provided with reinforcement material is also possible a cut to manufacture the desired rim culture, the cut being preferably made not through the reinforcement material, but laterally along the reinforcement material, but still within the elastomer layer.

The elastomer layers—both with and without reinforcement material—are applied for example by screen printing, when in principle also other application techniques come into consideration, such as relief printing, gravure printing, rouleaux (gravure printing method in which engraved printing rollers apply the elastomer on the fabric), spraying, application by nozzles or the like. The elastomer layers can also be applied by a dosing system.

Further advantages and expedient embodiments can be learned from the additional claims the figure description and the drawings. The figures show:

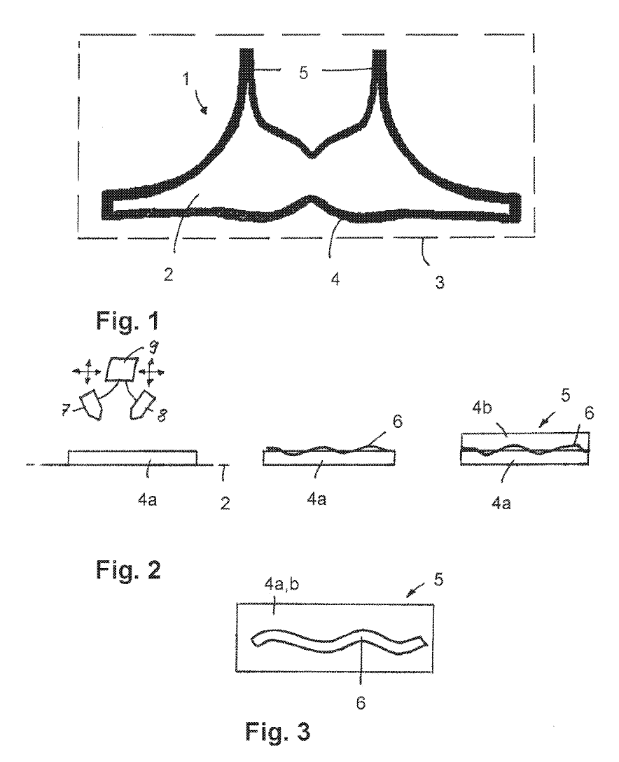

FIG. 1 shows a schematic view of a brassiere whose support is made of a composite of elastomer which is applied to a fabric layer, and reinforcement material.

FIG. 2 shows a side view of the combination of elastomer and reinforcement material in various stages of production.

FIG. 3 shows a top view of the combination of elastomer and reinforcement material.

In the figures, identical components are provided with the same reference numerals.

FIG. 1 shows schematically a brassiere as a garment 1 comprising one or more layers of fabric 2 and is encased in the rim region by an elastomer layer 4. The fabric layer 2 or the fabric layers have before cutting for example a rectangular basic format 3, within which the elastomer is applied in the track-like layers 4 in such a manner that the rim contour of the brassiere is formed. The elastomer layer 4 has a width of for example 2 mm to 20 mm. After the elastomer dries, the cutting is done by cutting the track-like elastomer layer 4 within the rim contour.

The supports 5 are also formed by an elastomer layer 4 forming the rim contour. However, in the area of the support 5 is introduced a reinforcement material 6 in the elastomer (FIG. 2, 3), which gives the supports 5 a significantly higher tensile strength. The reinforcement material 6 in particular consists of a plastic material, such as PLA or ABS, and is completely enveloped by the elastomer material.

FIG. 2 shows three different steps in the manufacture of the support 5 from the combination of elastomer layer 4 and reinforcement material 6. As can be recognized in the left picture of FIG. 2, in a first step, elastomer layer 4 is first applied to the fabric layer 2. To this end, the elastomer is advantageously heated so that the elastomer becomes deformable, in particular spreadable.

In a second step, which is shown in the middle picture of FIG. 2, subsequently the reinforcement material 6 is applied in liquid or at least soft, deformable state onto the first elastomer layer 4a. The application of the plastic material is done in one layer or in several layers, for example using a 3-D printing head, where it is also possible to apply the plastic material, for example, using a spray gun or a heated nozzle. The reinforcement material 6 has a relatively high temperature, for example at least 180° C. to ensure that the reinforcement material is in a liquid or at least soft, deformable state. As indicated in the figures, the reinforcement material 6 can be optionally applied in serpentine shape, namely both perpendicular to the plane of the fabric layer 2 and within the plane of the fabric layer 2 and transverse to the longitudinal extension of the support 5.

In a third, subsequent step, which is shown in the right picture of FIG. 2, a second, upper elastomer layer 4b is applied to the lower elastomer layer 4a, which completely envelops the reinforcement material 6. The application of the second elastomer layer 4b is preferably carried out after hardening of the reinforcement material 6.

In the left picture of FIG. 2 are shown two nozzles 7, 8, by which the elastomer layer 4 and the reinforcement material 6 are applied to the fabric layer 2. The nozzles 7, 8 can be adjusted by an adjustment mechanism in all three spatial directions and take up any position over the fabric layer. During the application of the elastomer layers 4a and 4b by the nozzle 7 and the reinforcement material 6 by the nozzle 8, the nozzles are moved along the intended track over the fabric layer 2; the elastomer material or the reinforcement material exit from the respective nozzle, and the discharge quantity of the elastomer or reinforcement material is controlled depending on the current nozzle position.

The position adjustment of each nozzle 7, 8 as well as the quantity of the reinforcement or elastomer material exiting through the respective nozzle 7, 8 is done by means of control signals of a control unit 9, wherein the control signals are generated on the basis of a digital template.

FIG. 3 shows the top view of the support 5 with the reinforcement material 6 embedded within the elastomer, which is spaced from the lateral edges—seen across the longitudinal extent of the reinforcement material 6.

Alternatively to the illustrated serpentine-shaped embodiment of the reinforcement material 6 is also possible a straight or a curved, possibly a buckled execution of the reinforcement material 6. Preferably, the course of the reinforcement material 6 follows the course of the elastomer layer 4. It can be advantageous, to connect the reinforcement material 6 directly in the region of one end of the fabric layer, for example by mechanical means such as by sewing, to create a firm connection between the reinforcement material 6 and the fabric layer 2.

1. A method for producing garments or bandages having a fabric layer which is connected to a composite of elastomer and reinforcement material, wherein the composite is formed by applying on an elastomer layer the reinforcement material in liquid or soft, deformable state, which hardens after application.

2. The method according to claim 1, characterized in that the elastomer layer and the reinforcement material are applied by a respective nozzle, whose position is adjustable in three spatial directions, wherein the quantity of the elastomer material and reinforcement material exiting through the respective nozzle is in each case adjusted.

3. The method of claim 1, characterized in that the reinforcement material is applied in several layers.

4. The method of claim 3, characterized in that at least two superposed layers of reinforcement material differ in their width and/or height.

5. The method according to claim 1, characterized in that the nozzle, through which the reinforcement material is applied, is formed as a 3-D printing head.

6. The method according to claim 1, characterized in that the reinforcement material has a higher melting point than the elastomer.

7. The method according to claim 1, characterized in that the reinforcement material is completely enveloped by the elastomer layer.

8. The method according to claim 1, characterized in that on the elastomer layer is applied the second elastomer layer enveloping the reinforcement material.

9. The method according to claim 1, characterized in that the elastomer layer is applied directly to the fabric layer.

10. The method according to claim 1, characterized in that the elastomer layer is placed into a mold part and the reinforcement material is applied onto the elastomer layer in the molding.

11. The method according to claim 10, characterized in that the composite of elastomer and reinforcement material is connected after curing to the fabric layer.

12. The method according to claim 1, characterized in that several track-like elastomer layers with or without reinforcement material are applied onto the fabric layer, wherein the fabric layer is subsequently cut within the track-shaped elastomer layers corresponding to the edge contour of the garment or the bandage.

13. The method according to claim 12, characterized in that the track-shaped elastomer layers are applied onto precisely one fabric layer.

14. The method according to claim 12, characterized in that the track-shaped elastomer layers are applied onto a fabric layer and then a second fabric layer is applied.

15. The method according to claim 1, characterized in that both elastomer layers with reinforcement material and elastomer layers without reinforcement material are applied onto the fabric layer.

16. The method according to claim 1, characterized in that the elastomer with or without reinforcement material is provided with flock coating.

17. The method according to claim 1, characterized in that the reinforcement material is made of a plastic material such as PLA (polylactide) or ABS (acrylonitrile-butadiene-styrene copolymer).

18. The method according to claim 1, characterized in that a thermoplastic elastomer is used.

19. The method according to claim 1, characterized in that a silicone is used as the elastomer.

20. The method of claim 2, characterized in that the reinforcement material is applied in several layers.
